(19) United States
(12) Patent Application Publication
(10) Pub. No.: US 2013/0084326 A1
(43) Pub. Date: Apr. 4, 2013

(54) LIPOSOMES FOR THE ORAL DELIVERY OF THERAPEUTIC AGENTS

(71) Applicants: Bruce Howe, Solana Beach, CA (US); Brian Keller, Antioch, CA (US)

(72) Inventors: Bruce Howe, Solana Beach, CA (US); Brian Keller, Antioch, CA (US)

(21) Appl. No.: 13/251,860

(22) Filed: Oct. 3, 2011

(51) Int. Cl. A61K 9/127; A61K 9/133; A61K 31/70; A61K 31/7088; A61K 38/02

(52) U.S. Cl. 424/450; 514/44 R; 514/1.1; 514/23

(57) ABSTRACT

The invention relates to liposomes having membranes composed of amphipathic molecules and long chain lipids in ratios that are proportionately higher in amphipathic molecules. Such ratios enhance the enzymatic breakdown of the inventive liposomes thereby improving the bioavailabity of encapsulated substances for oral delivery. Compositions of liposomes and methods of their use and manufacture are within the scope of the invention.

### BACKGROUND

The invention is in the field of liposomes. More particularly, the invention relates to liposomes for the oral delivery of therapeutic agents.

### SUMMARY OF THE INVENTION

Liposomes are spherical particles that encapsulate a fraction of a solvent (e.g. water), in which they freely float. Liposomes have one (unilamellar liposomes), or multiple concentric membranes (multilamellar liposomes). While conventional liposomes are constructed of amphiphilic lipids like phospholipids, some are composed of non-polar lipids like diacyl glycerol and polyethylene glycol conjugated lipids (“PEG lipids”). PEG lipids are commonly employed for steric stabilization of liposomes. When added in high concentrations PEG-lipids induce formation of mixed micelles, and depending on the lipid composition of their environment, these may adapt either a discoidal or a long threadlike shape. While effective in stabilizing liposomes, PEG lipids are associated with some undesirable side effects including bloating and intestinal discomfort.

Phospholipids are characterized by having a lipophilic and hydrophilic group on the same molecules. Upon interaction with water, polar lipids form self-organized bi-layer sheets, and when energy is added in the form of processing energy, spherical colloidal particles or lipid bodies will form.

Colloidal structures in pharmaceutical dosage forms are well known. There is a growing body of evidence that shows that the colloidal orientation of a dosage form can greatly contribute to the effectiveness of a particular drug or ingredient used to treat a myriad of diseases from cancer to headaches. One simple example is surfactants. Surfactants are used to combine lipophilic and hydrophilic components. Surfactants typically comprise a single chain of carbon atoms which are attached to a polar head group, with the carbon chain ranging from C-8 to C-28. Surfactants form micelles in an aqueous solvent which assist in comingling oil and water phases and drug solubility.

Examples of colloidal structures include polar lipids which have bulkier hydrophobic parts that cannot associate into micelles having a high curvature radii. Polar lipids therefore form bilayers which can self-close into liposomes or lipid vesicles when treated properly. A cross-section of a liposome (FIG. 1) depicts the hydrophilic heads of the amphophile orienting towards the water compartment and the lipophilic tails orient away from the water towards the center of the vesicle, thus forming a bilayer membrane surrounding and separating the water compartments. Consequently, water soluble compounds are entrapped in the water compartment and lipid soluble compounds are entrapped in the lipid section. Uniquely, liposomes can encapsulate both hydrophilic and lipophilic materials.

The lipid components that make up the membrane of liposomes are critical for the formation, stability, and eventual degradation of the liposomes. Various phospholipids and mixtures of phospholipids, sterols, cations, anions and the like have conventionally been used to adjust the liposome membrane for better stability, entrapment efficiency of specific encapsulates, skin permeation, and survival in the biological milieu.

Loading drugs and active ingredients into liposomes presents unique pharmaceutical and physical challenges. Special loading techniques have been employed for specific active ingredients to enable entrapment and increase concentrations within the liposomes. A great deal of scientific effort has been spent on this phase of liposomal development. However, the release of the drug from the liposomes presents at least as difficult a challenge if not more of a concern than entrapment. In some cases the encapsulated drug is delivered to the desired site but is inactive because it remains encapsulated. This is undesirable because in most therapeutic applications, high concentration of drug (for example) is required immediately, or in a short burst, making release of the drug critical. One example is the treatment of bacterial infections which normally require a large minimal inhibitory concentration (MIC) of antibiotic.

Lipid vesicles are broken down in vivo by two types of enzymes: lipases and esterases. The mechanism by which these enzymes degrade the vesicle membrane is the cleavage of the C1-C2 covalent bond on the glycerol backbone which faces the exterior of the vesicle. Lipases and esterases are able to target this bond and cleave it. However, in the case of membranes made with phospholipids, the phosphate ‘head’ creates a stearic interference to the enzymatic cleavage of the bond which inhibits the breakdown of the vesicle and the release of its encapsulated agents. What is needed in the art therefore are liposomes capable of enhanced enzymatic breakdown and greater bioavailability without the use of PEG lipids which have undesirable side effects. The invention overcomes these problems by providing a liposome having a membrane comprised of a mosaic of phospholipids and free fatty acid alcohol (e.g. diglyceride) which are free of a polar head group.

### BRIEF DESCRIPTION OF THE DRAWINGS

FIG. 1 is a depiction of a multilamellar liposome.

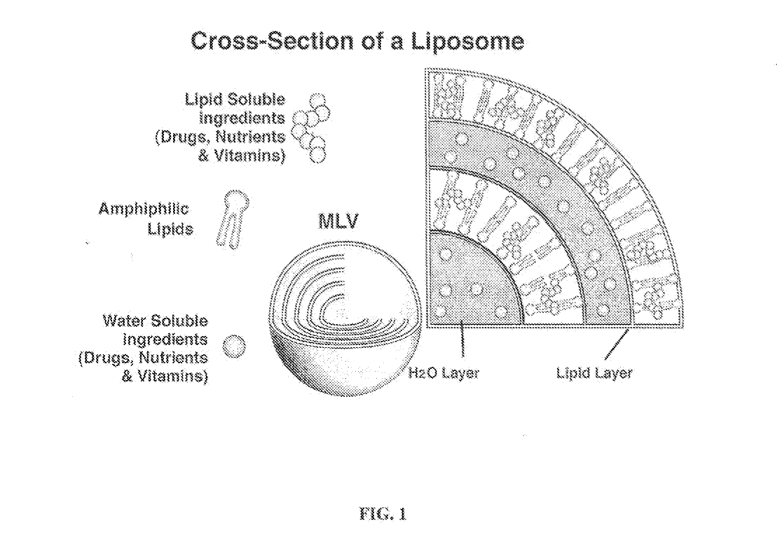

FIG. 2 is a chemical diagram of phosphotidyl choline.

FIG. 3 is a diagram of the chemical structures of two isomers of diacylglycerol.

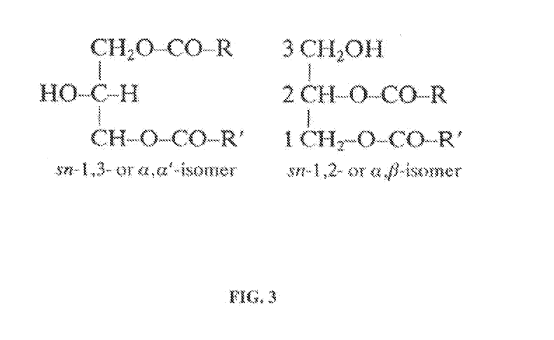

### DETAILED DESCRIPTION

The present invention relates to liposomes having improved enzymatic breakdown and bioavailability. The liposomes of the invention find use in compositions and methods where the oral delivery of therapeutic agents, including nutrients and drugs, is desired.

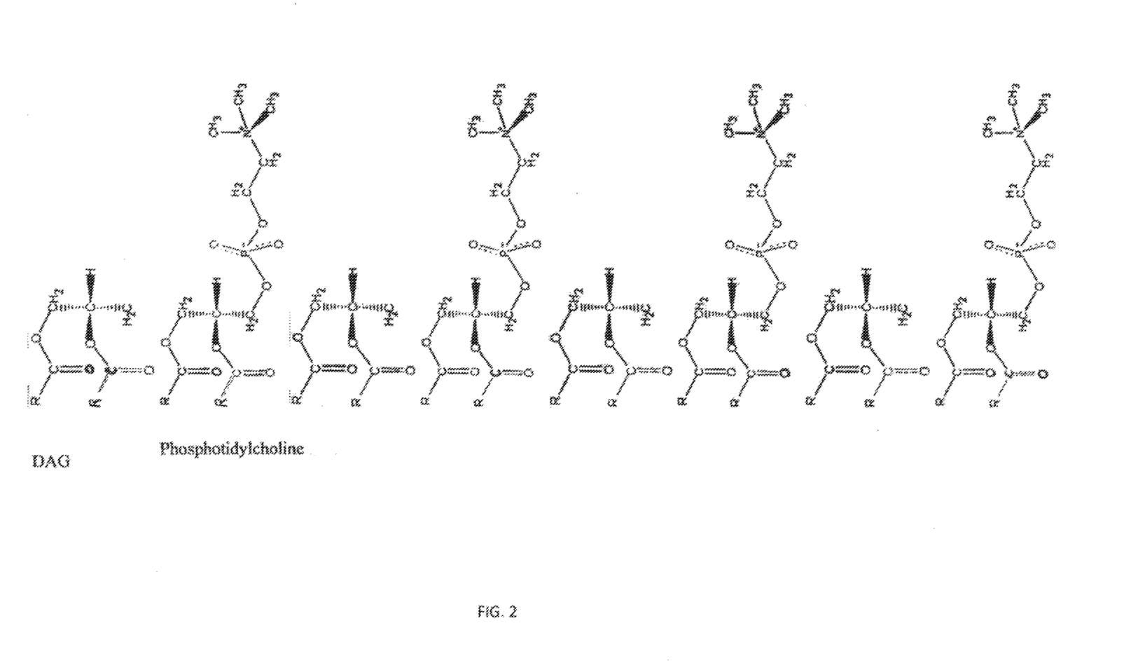

Without being limited to any particular theory, the liposomes of the invention achieve greater enzymatic lability by modulating the ratio of phospholipids to diacylglyceride molecules. In some aspects of the invention, the ratio of phospholipid to diacylglyceride molecules are equal or proportionately higher in phospholipids. This produces membranes comprised of diaclyglycerol molecules that are free of a polar head group. Because the diglyceride molecules do not have a head group attached to the glycerol backbone (see FIG. 2), there is no stearic or other interference and the preferred cleavage site (the C1-C2 bond) is exposed to the exterior environment and enzymatic breakdown. The liposomes of the invention therefore provide for the delivery of encapsulated materials (i.e. bioactive agents) in a certain and timely manner. In addition to the diacylglycerides providing the optimum geometry to intercolate into the membrane, they also have the added advantage of stabilizing the membrane in which they are formed.

The invention may be practiced with any phospholipid to diacylglyeride ratio that increases enzymatic access to the C1-C2 cleavage site. In one aspect of the invention, the phospholipid to diacylglyceride ratio is proportionately higher in diacylglyceride molecules. In one non-limiting embodiment, the molecular ratio of phospholipids to diacylglycerides is between about 100:1 to 2:1, respectively.

As used herein, the term “phospholipid” refers to a molecule having a hydrophilic polar head group comprised of one or more phosphate groups, and a hydrophobic tail comprised of two fatty acyl chains. In an aspect of the invention, fatty acyl chains for making the phospholipids of the invention are derived from diacylglycerols (also known as diglycerides, or “DAGs”). A diacylglycerol is a glyceride consisting of two fatty acid chains covalently bonded to a glycerol molecule through ester linkages. FIG. 2 depicts two isomeric forms of DAG. The invention may be practiced with either isomeric form, or combinations of such forms.

Diacylglycerol is a natural component of various edible oils, including palm oil, walnut oil and kikuie nut oil. DAGs for use with the invention may contain an even number of carbon atoms, typically between about 14 and 24 atoms. In an aspect of the invention, DAGs for use with the invention are about 16- or 18 carbon atoms in length. In another embodiment, the length of the DAG is 18 carbon atoms in length. The invention may be practiced with diacylglycerol molecules of any length, or combination of lengths, that permit the diacylgycerol molecules to form vesicles for the encapsulation of therapeutic agents as disclosed herein.

DAGs for use with the invention may be saturated and/or unsaturated, with the configuration of the double bond cis being preferred. One skilled in the art will appreciate that a desired level of membrane fluidity for the liposomes may be obtained by varying the length and level of saturation of the fatty acid chains of the DAG molecules, with shorter-chain fatty acids, and ones with greater unsaturation, providing greater membrane fluidity than longer, saturated fatty acid chains.

Although the liposomes of the invention are described as being derived from phospholipids containing diacylglyceride (DAG), one skilled in the art will appreciate that other phospholipids may be used. For example, liposomes may be formulated from phospholipids including, but not limited to, phosphatidylcholine, phosphatidylserine, phosphatidylethanolamine, phosphatidylinositol, and combinations thereof. It is also contemplated that the liposomes may be formed from amphipathic molecules that do not have a phosphate-based polar had group. For example, the liposomes of the invention may be formulated from sphingolipids, glycosphingolipids, ceramides and combinations thereof. All the compounds (and combinations of compounds) disclosed in this paragraph may be used alone, or in combination with, phospolipids.

In some aspects of the invention, the liposomes of the invention comprise other compounds for stabilizing the membrane to allow the liposome to resist gastrointestinal degradation. Such compounds include, but are not limited to, sterols. Sterols are large steroid-like molecules which have moieties that protrude from the surface of vesicles without a clear site of enzymatic attack. As used herein the term “sterol” refers to any of the various solid steroid alcohols widely distributed in plant and animal lipids (i.e. phytosterols and zoosterols).

Suitable sterols for use with the invention include, but are not limited to, cholesterol analogues which retain the ability to modulate membrane fluidity, including those disclosed in Gimpl, G., et al. (1997) Biochemistry 36:10959-10974, the disclosure of which is incorporated herein by reference. The liposomes of the invention may comprise, for example, cholesterol, sitosterol, campesterol, desmosterol, fucosterol, 22-ketosterol, 20-hydroxysterol, stigmasterol, 22-hydroxycholesterol, 25-hydroxycholesterol, lanosterol, 7-dehydrocholesterol, dihydrocholesterol, 19-hydroxycholesterol, 5α-cholest-7-en-3β-ol, 7-hydroxycholesterol, epicholesterol, ergosterol, dehydroergosterol, and combinations thereof. One skilled in the art will appreciate that membrane fluidity and digestibility may be adjusted to desirable levels by varying the phospholipid:diacylglycerol:sterol ratio, as well as the type of molecules (e.g. phospholipids, diacylglycerols and sterols) that are selected.

The liposomes of the invention can also be mechanically stabilized using certain phospholipids, e.g. phospholipon 90H, and cholesterol in an optimum molar ratio of 2:1

One aspect of the invention concerns the size of the liposomes. Suitable size ranges for the liposomes of the invention include, but are not limited to, liposomes that are between about 20 nm and 1,000 nm in diameter.

In aspects of the invention, the liposomes are formulated for the oral delivery of one or more bioactive agents. As used herein, the term “bioactive agent” refers to substances that have an effect on a biological system. Bioactive agents include, but are in no way limited to, vitamins, minerals, proteins, nucleic acids, amino acids, carbohydrates (e.g. polysaccharides and monosaccharides), pharmaceuticals, phytonutrients, and combinations thereof. In one non-limiting embodiment of the invention, the liposomes are used to encapsulate vitamins and/or minerals (e.g. phytonutrients). The liposomes of the invention may also be used to encapsulate, for example, L-glutathione, green tea (e.g. green tea extract), quercitin, turmeric, coenzyme Q10, resveratrol, grape seed extract, lycopene, lutein, astaxanthin, vitamin D, and combinations thereof. As used herein, the term “encapsulate” refers to the enclosing of a material within at least one phospholipid/DAG membrane of a liposome. Bioactive agents for use with the invention may be encapsulated inside the bilayered membrane, or between two bilayered membranes of a multilamellar liposome.

Another aspect of the invention concerns the source material from which the phospholipids and the diacylglycerol molecules are derived. As known in the art, phospholipids for the manufacture of the inventive liposomes may be derived from natural sources, or “source materials,” such as animal sources (e.g. egg yolk) and/or plant sources (e.g. soy beans). In an aspect of the invention, the source materials are non-genetically modified (i.e. free of recombinant DNA).

The liposomes of the invention may be free of, or essentially free of, polyethylene glycol conjugated lipids (“PEG lipids”). PEG lipids are known to be associated with disorders including, but not limited to, nausea, bloating, gas, diarrhea and abdominal discomfort (hereinafter “digestive side effects”). Thus, in one aspect of the invention, liposomes are made according to formulations that are free of, or essentially free of, PEG lipids. As used herein, “essentially free of PEG lipids” means that a liposome contains a level of PEG lipids that does not produce a digestive side effect in a subject when the subject ingests an amount of the liposome necessary to achieve a desired nutritional or pharmaceutical effect.

Although the invention has been disclosed as set forth above, one skilled in the art will appreciate that the liposomes of the invention may be made and practiced with other materials and methods that are similar in composition and/or effect to those described herein without departing from the scope of the invention.

We claim:

1. A liposome comprising:
a. phospholipids; and
b. diacyl glycerol molecules;
c. wherein the phospholipids are present in a ratio that is equal or proportionately higher in phospholipids

2. The liposome of claim 1, wherein the molecular ratio of phospholipids to diacyl glycerol molecules is between about 100:1 to 2:1.

3. The liposome of claim 2, wherein the phospholipids are substituted with a non-phosphate-based amphipathic molecule selected from the group consisting of sphingolipids, glycosphingolipids, ceramides and combinations thereof.

4. The liposome of claim 2, further comprising a non-phosphate-based molecule selected from the group consisting of one or more sphingolipids, glycosphingolipids, ceramides and combinations thereof.

5. The liposome of claim 2, 3 or 4, further comprising a bioactive agent selected from the group consisting of vitamins, minerals, proteins, nucleic acids, amino acids, carbohydrates, pharmaceuticals, and combinations thereof.

6. The liposome of claim 5, wherein the bioactive agent comprises at least one vitamin and/or at least one mineral.

7. The liposome of claim 6, wherein the bioactive agent is a phytonutrient.

8. The liposome of claim 6, wherein at least one of the phospholipids and diacylglycerol molecules are derived from a non-genetically modified source material.

9. The liposome of claim 6, wherein the liposome is essentially free of PEG lipids.

10. The liposome of claim 2, 3 or 4, wherein the liposome is unilamellar or multilamellar.
